(19) United States
(12) Patent Application Publication
(10) Pub. No.: US 2009/0241967 A1
(43) Pub. Date: Oct. 1, 2009

(54) MANDIBULAR AND NECK SUPPORT AND POSITIONING DEVICE

(76) Inventor: Walter Gerardo ORENCEL, Buenos Aires (AR)

Correspondence Address: LOTT & FRIEDLAND, P.A., P.O. BOX 141098, CORAL GABLES, FL, 33114-1098, US

(21) Appl. No.: 12/057,108

(22) Filed: Mar. 27, 2008

(51) Int. Cl. A61B 19/00

(52) U.S. Cl. 128/845

(57) ABSTRACT

A mandibular and neck support and positioning device intended to keep the airways unobstructed, facilitating breathing in patients with trauma or under anesthesia, on sedatives or in deep relaxation, where the device has a base support for at least the patient's neck, the base support has at least two pusher parts located on the sides to lift the mandible and, once fixed, said side pieces remain in place whenever a new position needs to be adopted.

### TECHNICAL FIELD

This invention relates to the field of devices and appliances used in medicine and emergency care and, more specifically, to a mandibular and neck support and positioning device to keep airways unobstructed, facilitating breathing in patients with trauma or during surgeries and treatments under anesthesia or sedatives, or with the patient in deep relaxation.

### BACKGROUND OF THE INVENTION

In medical and emergency care, it is often necessary to assist or improve lung ventilation in patients who, for example have breathing difficulties under anesthesia or sedatives, or may be unconscious or have their muscles relaxed.

There are currently a variety of systems and maneuvers to help patients who are unconscious, relaxed, sedated and/or under general anesthesia breathe more easily.

These systems provide supplementary or oxygen-enriched air and devices to ensure the air reaches the patient's lungs in the best possible way.

In turn, these maneuvers enable the mobilization and positioning of certain body parts, more specifically the head, making the upper airway more permeable and improving air intake.

Generally, patients who have been subject of surgical procedures or examinations under anesthesia, or are recovering from an intubation period, require the attention of a qualified person, usually a nurse, physician or anesthesiologist, who should perform maneuvers or apply techniques in order to keep adequate lung oxygenation for a certain period of time, until the person recovers from respiratory depression.

Such respiratory depression occurs, for example, when a patient subject to anesthesia relaxes their muscles, including those related to the respiratory system and the airway through which breathing air flows.

In particular, such a state of muscular relaxation is observed in the neck and mandibular muscles, making the mandible go down or drop and reducing the breathing capacity of the anesthetized person.

In general, a person under anesthesia, on sedatives, in relaxation or in an unconscious state is in supine position on an often horizontal surface, such as a bed, stretcher, or even the floor as is the case with an accident warranting on-site care.

The most frequently used maneuvers or techniques to improve a person's oxygenation are the following:

The use of an oropharyngeal tube or cannula, or Mayo tube, to move the posterior tongue forward, preventing the airway from being obstructed. In this case, the size of the airway tube should be specially taken into account; otherwise, the objective might fail to be achieved and the tongue might move further backwards. This technique is generally used while the patient is unconscious to facilitate mask ventilation, because if used during the conscious period it could stimulate nausea or “gag” reflexes. In addition, the tube does not prevent the glottic closure caused by the posterior movement of the hyoid bone, so placing it without prior head hyperextension and mandibular subluxation does not fully permeabilize the airway.

Another method is leaving the laryngeal mask and/or endotracheal tube on until the person recovers effective ventilation. This requires the prior use of a laryngeal mask and/or endotracheal tube, and should be avoided in case of mild anesthesia or short procedures not warranting such invasive methods.

It is worth mentioning that both methods involve the use of tubes and/or masks which are introduced through the mouth to the larynx or trachea; therefore, intubated patients may suffer a subsequent discomfort or burning, or even a traumatic experience if they recover consciousness at least partially while intubated.

Finally, other maneuvers of interest are those known as “head hyperextension” and “mandibular subluxation.”

“Head hyperextension” consists of tilting the head backwards with the aim of facilitating air intake, and “mandibular subluxation” consists of placing two or three fingers under each side of the lower mandibular angle, causing traction on and elevating it, in order to avoid the lowering of the mandible and soft parts surrounding it.

Both maneuvers are performed by one person, without any special accessory devices.

Although in the above maneuvers the neck could be partly supported with the help of a pillow, the person performing it must, by manipulation, keep the mandible in the correct position for a certain period of time, which usually results in a technically difficult and exhausting task.

This person would also be unable to carry out any other manual activities, precisely when drug administration and monitor control is necessary.

While “head hyperextension” and “mandibular subluxation” are very effective, even in combination with other maneuvers, their application is usually difficult since there is no device capable of emulating such maneuvers.

It would therefore be useful to use a mandibular and neck support and positioning device to emulate the “mandibular subluxation” maneuver and, optionally, the “head hyperextension” maneuver, facilitating the application of both techniques for extended periods of time without requiring constant attention by a care provider.

### SUMMARY OF THE INVENTION

It is an object of the present invention to provide a mandibular and neck support and positioning device so as to emulate the “mandibular subluxation” maneuver, that is, lifting the mandible to facilitate the opening of the upper airway.

It is another object of the present invention to provide a mandibular and neck support and positioning device so as to keep the mandible in the desired position without the need for another person to intervene.

It is yet another object of the present invention to provide a mandibular and neck support and positioning device so as to support the neck, preventing the head from moving to the sides.

It is still another object of the present invention to provide a mandibular and neck support and positioning device so as to, where appropriate, tilt the head back emulating the “head hyperextension” maneuver.

It is yet another object of the present invention to provide a mandibular and neck support and positioning device that is easily-operated, small enough to be transported and used in a fixed position, or over a stretcher and other moving surfaces.

It is still another object of the present invention to provide a mandibular and neck support and positioning device to emulate at least the mandibular subluxation maneuver, of use to keep the patient's upper airways unobstructed when the patient is in supine position. This device comprises a base support for at least the patient's neck, having at least two lateral pusher parts.

These and other objects, features, and advantages of the present invention may be more clearly understood and appreciated from a review of ensuing detailed description of the preferred and alternate embodiments and by reference to the accompanying drawings and claims.

### BRIEF DESCRIPTION OF THE DRAWINGS

To better understand the object of the present invention, several drawings representing one embodiment for the present invention are provided by way of example, wherein:

FIG. 1 is a perspective view of the device of the present invention, showing a person's head and neck in hidden lines, according to an embodiment of the present invention.

FIG. 2 is a perspective view of the device of the present invention, having tilted the patient's head backwards, according to an embodiment of the present invention.

FIG. 3 is a perspective view of the device of the present invention, having moved the patient's mandible with the side pusher pieces, according to an embodiment of the present invention.

### DETAILED DESCRIPTION OF THE INVENTION

While the present invention will be described more fully hereinafter with reference to the accompanying drawings, in which an embodiment of the present invention is shown, it is to be understood at the outset of the description which follows that persons of skill in the appropriate arts may modify the invention herein described while still achieving the favorable results of this invention. Accordingly, the description which follows is to be understood as being a broad, teaching disclosure directed to persons of skill in the appropriate arts, and not as limiting upon the present invention.

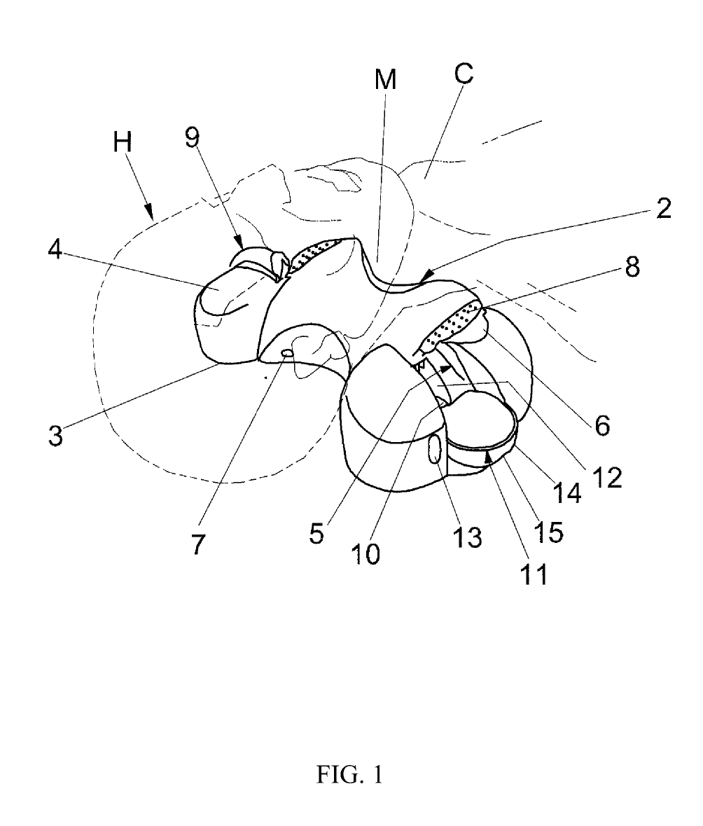

Referring to FIG. 1, the invention comprises a base support 1 for the patient's neck C that may act as a pillow to rest it. Particularly, the neck C rests upon the neck central support 2.

Preferably, the geometry described in the figures has been adopted for said central support 2, so as to coincide with the shape of the neck, enabling its better adjustment to the physical build of a greater range of people.

This base support 1 has an inferior support surface 3, generally flat and preferably made of an anti-skid material or texture to safely place and position the device on any surface. It is worth mentioning that this device is portable and can be used in several surfaces, such as floors, stretchers, beds, and similar surfaces.

The base support 1 is fitted with protruding lateral stops 4, located to both sides of the head “H”, that is, at both ends of the base support 1.

The lateral stops 4 help to prevent the head “H” from moving to the sides. Furthermore, the base support 1 fitted with the lateral stops 4 will have, as seen in FIG. 1, side slots 5 leaving a free space to slide or position other components described below, and side grooves 6 for the person assisting the patient to place their hands in order to move the device as needed.

The device has been particularly fitted with two side slots 5 at each side of the base support 1 and fully cutting through the side stoppers 3. These side slots 5 can be shaped in other ways, for instance, a slot that does not reach to the lower support surface or is somewhat inclined to one of the sides, among others.

Similarly, the configuration of lateral stops 4 may, with removable and/or articulated stops, or even multiple stops, where the side slots 5 would be determined by the free space between two nearby lateral stops 4.

In the embodiment shown in FIGS. 1-3, the central support 2 is made movable through joints 7.

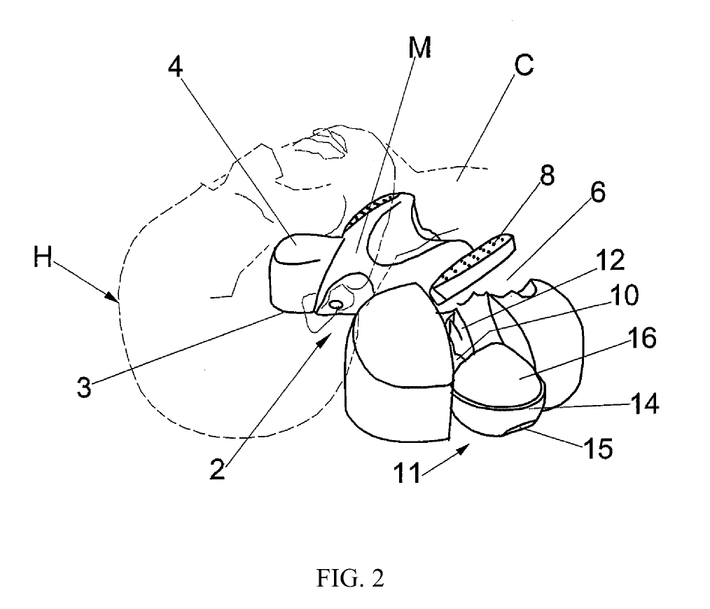

Referring to FIG. 2, the central support 2 can rotate together with the neck “C” and head “H” so as to emulate the “head hyperextension” maneuver, that is, lifting the neck until the head is tilted back to favor the passage of air. This rotation, however, is optional, and the central support 2 can rotate in every direction.

In order to facilitate the operation of the central support 2, the latter has been fitted with grips 8, located to the sides and coinciding with grooves 6, thus enabling the person operating the device to safely access to, hold, and operate said central support 2 with the aim of tilting the head back as described above.

Since the joints 7 are aimed at tilting the head “H” back when the person is in supine position, they can be located symmetrically or asymmetrically to the central support 2, that is, they can located in such a way that the central support 2 is made to rotate upon a symmetrical center and the device can be used reversibly. Furthermore, the device may be fitted with a round or another skid system for the central support 2 to tilt the head “H” backwards.

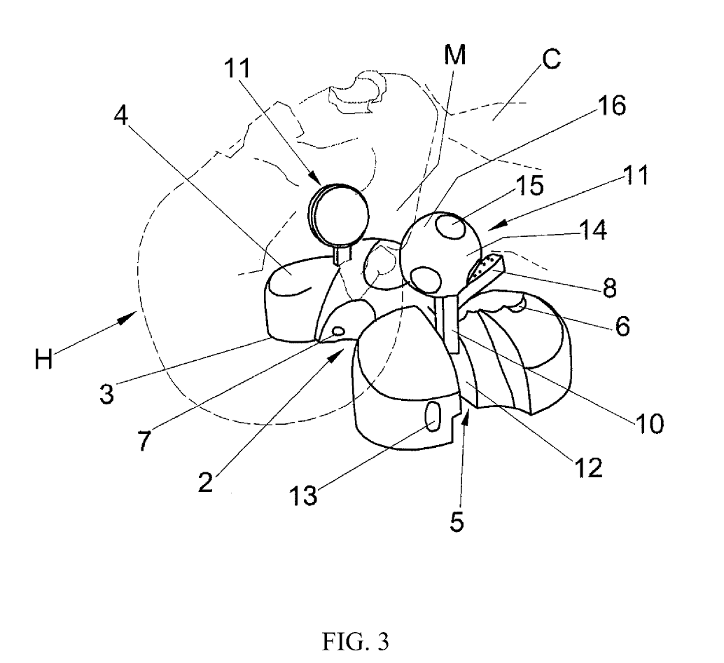

In reference to FIG. 3, the free space provided by the slots 5 holds a pair of lateral pusher parts 9 that rotate towards the main support 2, that is, the head “H”. In turn, each of the lateral pusher parts 9 includes a pivoting arm 10, which hinges on the sides of the base support 1. This arrangement of pivoting arm 10 allows same to slide through slot 5 towards the mandible

Each pivoting arm 10 has a protuberance 11, which exerts pressure on the mandible “M,” lifting it and thus emulating the “mandibular subluxation” maneuver that presently can only be performed manually.

It is worth noting that the device of the present invention autonomously maintains the position of the head and mandible. That is, it maintains the configuration of the central support 2 and the pivoting arms 10 that include the protuberances 11 supporting the mandible “M” without the need for a person to make a sustained effort on the moving parts of the invention. This is possible because the pivoting arms 10 have a binding 12 that enables them to adopt a fixed angle or position, especially one that allows for the exerting of pressure on the mandible “M” so as to prevent it from dropping due to muscular relaxation.

In one embodiment, this binding 12 has a serrated surface and a second surface that slides or turns in a single direction acting as a pawl; making it possible to release or immobilize the moving arm 10 at will, acting on a lever 13 or release/hold key, as preferred.

Other types of binding system, well known in the art, may be used. For example, a constructive-type lever, also known as fast-action lever, with the aim of pressing at least one fixed surface to the base support 1 on said pivoting arm 10, keeping it in its position by friction. These fast-action levers operate on the basis of an eccentric cam pressing at least two surfaces together when rotating, which increases friction force between the parts and therefore prevents their relative movement up to a certain effort limit.

It may therefore be said that bindings of this or another kind may be easily added, since the constructive features and the efforts to be endured by the moving parts enable so.

Referring to the embodiment in FIGS. 1-3, it can be seen that the protuberances 11, in turn, comprise a support sphere 14 that may also include some depressions 15 or bas-reliefs to improve the grip on the protuberances.

Said support sphere 14 is joined to the pivoting arm 10 in a fixed or removable way, and may both be placed in the end of the moving arm 10 and slide on it. In turn, said support sphere 14 contains a pressure pad 16 that will come into contact with the face and exert pressure on the mandible

Said pressure pad 16 may be made of a flexible material such as foam rubber, rubber, plastic, polyurethane, or a similar material, and also be coated with a laminar material such as leather, fabric, or synthetic materials. In addition, the embodiment made of a flexible material coated or uncoated by laminar material may extend to other components, such as the lateral stops 4 and the central support 2, thus increasing comfort for the patient.

Furthermore, if made of only one material or component, or a combination of materials or forms, protuberances 11 may be movable or fixed and/or selectable from a range of interchangeable protuberances. By way of example, both protuberances 11 may be made up of a flexible material sphere with a center opening that is threaded onto the end of pivoting arm 10, or said protuberance 11 may telescopically slide on to pivoting arm 10.

When the protuberances 11 are movable, they will have at least one binding to fix them to the end of pivoting arms 10; said bindings may be screws, fast-action levers, and/or similar devices.

Furthermore, both pivoting arms 10 and said protuberances 11 may form a single piece with the right size and proportion for particular physical builds. Alternatively, said protuberances 11 may slide through some type of skid system, thread and/or mechanism slide towards the mandible so as to lift it and thus emulate the “mandibular subluxation” maneuver.

Accordingly, it will be understood that one embodiment of the present invention has been disclosed by way of example and that other modifications and alterations may occur to those skilled in the art without departing from the scope and spirit of the appended claims.

I claim:

1. A mandibular and neck support and positioning device for a patient that maintains said patient's upper airway unobstructed through emulation of the mandibular subluxation maneuver while said patient is in the supine position, said device comprising:
a base support for said patient's neck; and
a plurality of lateral pusher parts

2. The device of claim 1, wherein said base support comprises a central support for said patient's neck and two or more lateral stops each disposed on each side of said central support.

3. The device of claim 2, wherein said central support is movably disposed on said base support.

4. The device of claim 3, wherein said movably disposed central support can pivot forward and backward in relation to said base support.

5. The device of claim 2, wherein said central support comprises at least two grips, each positioned on either side of said patient's neck.

6. The device of claim 3, wherein said central support comprises at least two grips, each positioned on either side of said patient's neck.

7. The device of claim 4, wherein said central support comprises at least two grips, each positioned on either side of said patient's neck.

8. The device of claim 2, wherein each of said pusher parts comprises a movable arm attached to said central support and at least one protuberance.

9. The device of claim 8 wherein each of said protuberances is movably mounted on said movable arm and may be temporarily fixed in at least one position on said movable arm.

10. The device of claim 8 wherein each of said movable arms is pivotally mounted on said base support and may be fixed at a definite angle of pivot.

11. The device of claim 9 wherein each of said movable arms is pivotally mounted on said base support and may be fixed at a definite angle of pivot.

12. The device of claim 8 wherein said protuberance is composed of a material selected from the group comprised of: foam rubber, rubber, plastic and polyurethane.

13. The device of claim 9 wherein said protuberance is composed of a material selected from the group comprised of: foam rubber, rubber, plastic and polyurethane.

14. The device of claim 10 wherein said protuberance is composed of a material selected from the group comprised of: foam rubber, rubber, plastic and polyurethane.

15. The device of claim 11 wherein said protuberance is composed of a material selected from the group comprised of: foam rubber, rubber, plastic and polyurethane.
